(19) United States
(12) Patent Application Publication
(10) Pub. No.: US 2013/0130286 A1
(43) Pub. Date: May 23, 2013

(54) METHODS FOR ASSESSING THE IMMUNE SYSTEM IN A PATIENT

(71) Applicant: THE TRUSTEES OF COLUMBIA UNIVERSITY IN THE CITY OF NEW YORK, New York, NY (US)

(72) Inventors: Samuel C. Silverstein, New York, NY (US); John D. Loike, Jamaica, NY (US); Sadna Budhu, Pelham, NY (US); Raphael Clynes, West Nyack, NY (US); Peter P. Lee, San Marino, CA (US)

(73) Assignee: THE TRUSTEES OF COLUMBIA UNIVERSITY IN THE CITY OF NEW YORK, New York, NY (US)

(21) Appl. No.: 13/728,697

(22) Filed: Dec. 27, 2012

Related U.S. Application Data
(63) Continuation of application No. 13/509,418.
(60) Provisional application No. 61/260,255, filed on Nov. 11, 2009.

(51) Int. Cl. G01N 33/68

(52) U.S. Cl. CPC G01N 33/68
USPC 435/7.92

(57) ABSTRACT

Methods of determining the onset or susceptibility of an immunological disease are provided herein. Also provided are immunoassay techniques for carrying out such methods.

### CROSS-REFERENCE TO RELATED APPLICATIONS

The present invention claims benefit to U.S. Provisional Application No. 61/260,255 filed Nov. 11, 2009, the contents of which are incorporated herein by reference.

### GOVERNMENT RIGHTS IN THE INVENTION

This invention was made with government support under Grant No. A120516 awarded by NIH-NIAID. The government has certain rights in the invention.

### FIELD OF THE INVENTION

The present invention generally relates to screening methods for evaluating the immune system.

### BACKGROUND OF THE INVENTION

Despite the rise of terrorist attacks worldwide, the 9/11 attack on New York City, and the creation of and investments in, the Department of Homeland Security, few U.S. medical facilities are prepared to screen victims of a nuclear accident or attack, and to discriminate between those in no immediate danger and those in need of immediate medical intervention. The bone marrow is one of the first targets of radiation injury. Exposure to sub-lethal irradiation causes destruction of lymphocytes (lymphopenia) within the first 24 hours. While this lymphopenia is itself not immediately life threatening, it is an indicator of significant radiation injury, and a harbinger of the potential for lethal complications in the next several days. These lethal complications include destruction of platelets (thrombocytopenia) which predisposes to severe bleeding, and destruction of neutrophils (neutropenia), which predisposes to lethal bacterial blood infections (sepsis).

In the event of exposure of a large number of people to radiation, medical facilities will be deluged with individuals who have survived the initial event and understand their potential need for supportive therapy in the days or weeks ahead. Obtaining blood to assess the number of white cells will only reveal those cells within the vascular system but not other areas of the body that contain immune such as the bone marrow, lymph organs, or the liver. By 24 hours after the event, a white blood count will likely be insufficient to distinguish individuals who have sustained significant radiation damage to their bone marrow from those who have not. By 48 hours after the event, and thereafter, it will be important to distinguish individuals who are neutropenic and are in danger of fatal sepsis, and those who are equally neutropenic but not in immediate danger of sepsis.

Chemotherapy is an important therapy for the treatment of most cancers. However, most chemotherapeutic agents will suppress the immune system as it destroys most dividing cells in the body besides the cancer cells. Clinicians are acutely aware of the importance of monitoring the immune status of a cancer patient during the repeated cycles of chemotherapy. As with radiation exposure, individuals on chemotherapy who develop neutropenia will be in danger of developing sepsis.

Huizinga et al. showed that the blood concentration of a protein released by neutrophils called sCD16b predicts with −90% accuracy individuals who will become septic absent medical intervention (3). A need exists for other predictive methods for determining the onset of, or susceptibility to, immunological diseases such as sepsis. A need also exists for analytical techniques for detecting the presence of biological substances that are predictive of, or indicative of a susceptibility to, immunological diseases.

### SUMMARY OF THE INVENTION

Among the various aspects of the present invention is the provision of a methods and techniques for detection of biological substances in biological patient samples for use in the diagnosis, prognosis, and monitoring of diseases. Efficient identification of biological substances aids in devising effective treatment strategies.

Briefly, therefore, the present invention is directed to an enzyme linked immunosorbent assay (ELISA) kit for determining the presence of soluble CD16b protein, which comprises at least one antibody specific to the protein to measure its molar ratio in biological sample of a neutropenic patient, and wherein said molar ratio is indicative of the onset or susceptibility of sepsis in the patient.

Another aspect of the invention is directed to a method of predicting the onset of sepsis in a neutropenic patient, the method comprising providing a biological sample from the patient; determining the presence of soluble CD16b protein in the biological sample; and predicting the onset of sepsis based on the presence or absence of soluble CD16b in the biological sample, wherein the determining is carried out using an ELISA kit as described herein.

Another aspect of the invention is directed to a method of detecting an increased susceptibility to sepsis in a neutropenic patient, the method comprising providing a biological sample from the patient; determining the presence of soluble CD16b protein in the biological sample; and predicting the onset of sepsis based on the presence or absence of soluble CD16b in the biological sample, wherein the determining is carried out using an ELISA kit as described herein.

Another aspect of the invention is directed to a method of predicting the onset of sepsis in a neutropenic patient receiving a cancer treatment, the method comprising providing a biological sample from the patient; determining the presence of soluble CD16b protein in the biological sample; and predicting the onset of sepsis based on the presence or absence of soluble CD16b in the biological sample.

Another aspect of the invention is directed to a method of detecting an increased susceptibility to sepsis in a patient receiving a cancer treatment, the method comprising analyzing a biological sample from the patient for the presence of soluble CD16b protein, wherein the presence or absence of soluble CD16b protein is indicative of an increased susceptibility to sepsis.

Another aspect of the invention is directed to a method of predicting the onset of an inflammatory disease in a neutropenic patient, the method comprising providing a biological sample from the patient; determining the presence of a neutrophilic surface protein in the biological sample; and predicting, based on said determining, the onset of the inflammatory disease in the neutropenic patient.

Another aspect of the invention is directed to a method of detecting an increased susceptibility to an inflammatory disease in a neutropenic patient, the method comprising analyzing a biological sample from the patient for the presence of a neutrophilic surface protein, wherein the presence of the neutrophilic surface protein is indicative of an increased susceptibility to the inflammatory disease.

Another aspect of the invention is directed to a method of predicting the onset of an inflammatory disease in a patient receiving a cancer treatment, the method comprising providing a biological sample from the patient; determining the presence of a neutrophilic surface protein in the biological sample; and predicting, based on said determining, the onset of the inflammatory disease in the patient.

Another aspect of the invention is directed to a method of detecting an increased susceptibility to an inflammatory disease in a patient receiving a cancer treatment, the method comprising analyzing a biological sample from the patient for the presence of a neutrophilic surface protein, wherein the presence of the neutrophilic surface protein is indicative of an increased susceptibility to the inflammatory disease.

Other objects and features will be in part apparent and in part pointed out hereinafter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a flow chart showing the steps of the methods of the present invention.

FIG. 2 illustrates a computer for implementing selected operations associated with the methods of the present invention.

FIG. 3 illustrates background information regarding neutrophils and Fcγ receptor type IIIb (FcγRIIIb, CD16b).

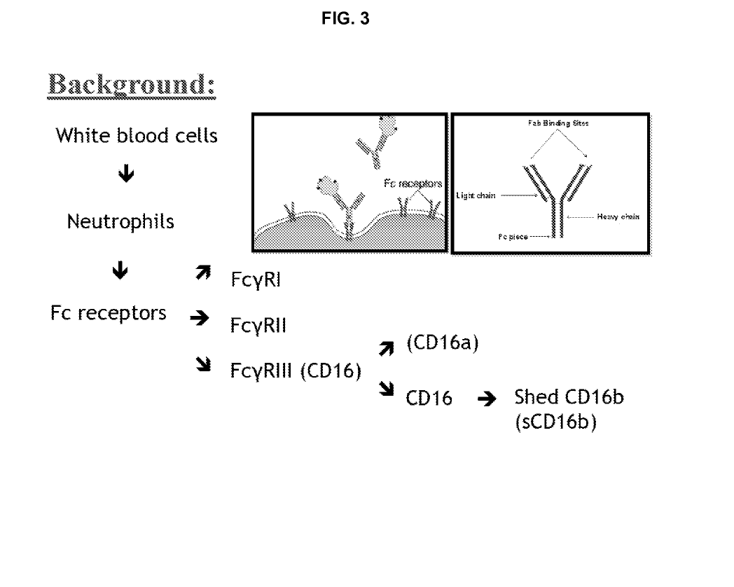

FIG. 4 illustrates an exemplary sandwich ELISA in accordance with certain embodiments described herein.

FIG. 5 depicts immunofluorescence staining of human neutrophils with antiCD16b antibodies; Staining cells with anti-CD16b antibodies. Freshly isolated human neutrophils were allowed to adhere to glass coverslips for 60 mins. The cells were fixed with acetone and formaldehyde and incubated with either control murine IgG or murine anti CD16b IgG for 15 mins. The cells were then washed with phosphate buffered saline and further incubated with the Alexa Fluor goat anti-mouse IgG for 20 mins and washed again in phosphate buffered saline. Fluorescence stained cells were then observed under the microscope.

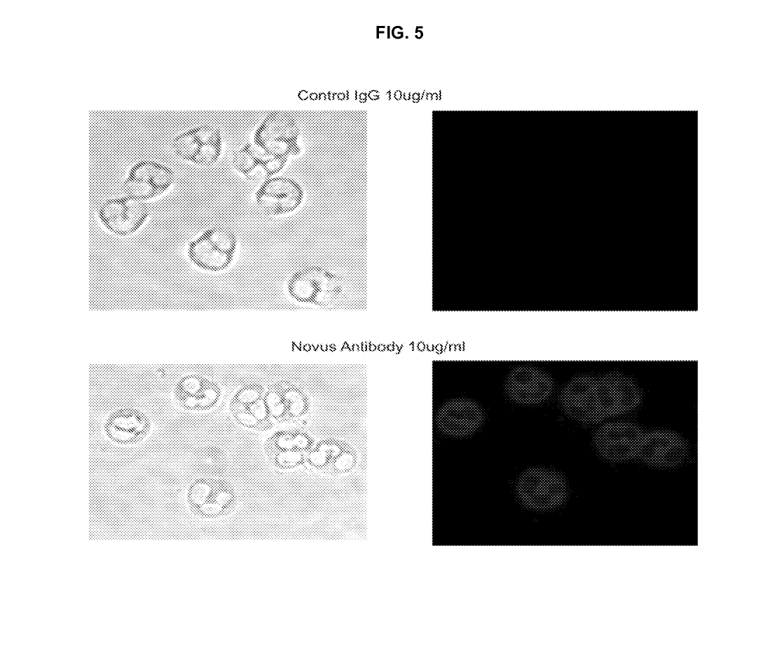

FIG. 6 depicts an ELISA Assay for human CD16b; Using the sandwich ELISA, fluorescence was measured for known concentrations of pure recombinant human CD16b. This illustrates the Standard Curve of Fluorescence vs. CD16b Concentration. Various concentrations of recombinant human CD16b was added as the indicated concentration to 96-well ELISA plates that were was coated with mouse anti-human CD16b antibody. After a one hour incubation, a room temperature biotinylated goat anti-CD16b diluted 1:500 in block was added for 1 hour before the sequential addition of HRP-labeled avidin and Amplex Red. The wells were then real on a CytoFluorII fluorescence plate reader.

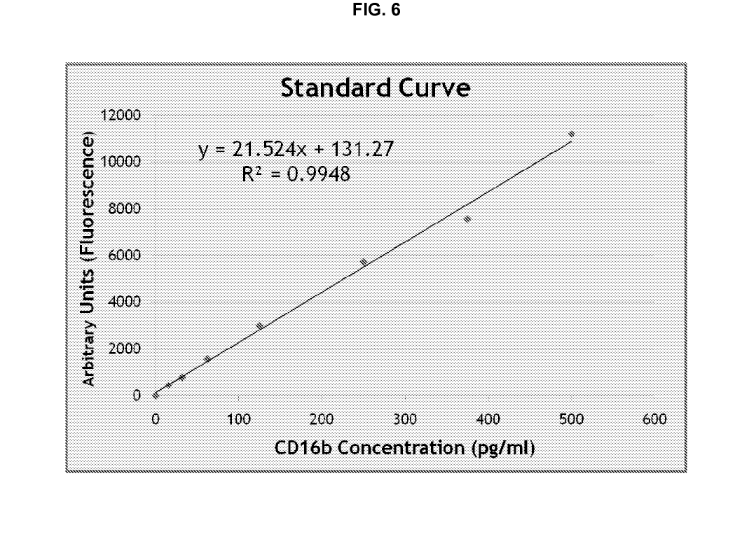

FIG. 7 depicts the spontaneous shedding of CD16b by control and fMLP-treated human neutrophils; CD16b Shedding by 10⁶ Human Neutrophils: The Effect of Varying Time of fMLP Incubation. 10⁶ human neutrophils were added to eppendorf tubes in suspension in the presence or absence of 10⁻⁷ M fMLP for various times. The neutrophils were placed on a shaker at 200 rpm for 0, 30, 60, or 90 minutes. The neutrophils were pelleted, and the supernatural removed and assayed for CD16b using the ELISA assay. Formylmethionine-leucine-phenylalanine (fMLP), a tripeptide released by bacteria and a stimulant of neutrophil migration, induces freshly isolated human neutrophils to shed.

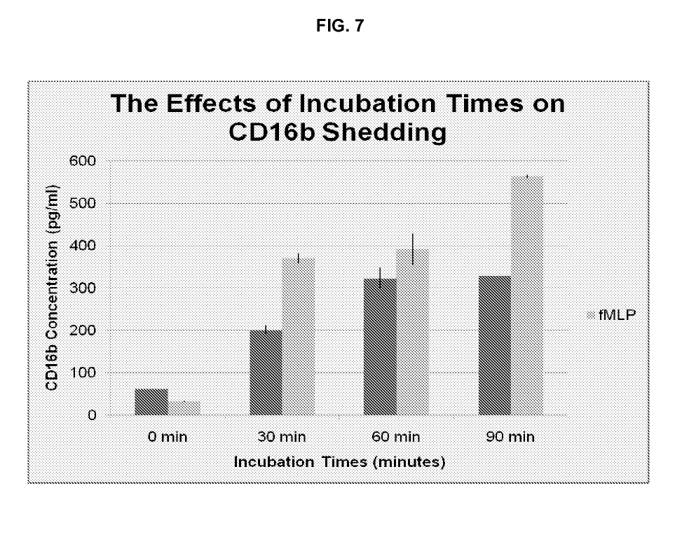

FIG. 8 illustrates the effects of various chemoattractants on sCD16b shedding by human neutrophils allowed to adhere to fibrinogen-coated surfaces; Effects of chemoattractants on sCD16b at varying neutrophil concentrations. Indicated concentrations of human blood derived neutrophils were allowed to adhere to 96 well plates pre-coated with fibrinogen for 90 min. Neutrophils were incubated with the following cytokines or chemoattractants. PMA (10⁻⁹M ), fMLP (10⁻⁷M), or LTB4 (10⁻⁷M). The supernatant was collected from each sample and assayed for sCD16b content. With increasing concentrations of neutrophils, more sCD16b was detected in human plasma. Incubation of 10⁻⁵ and 2×10⁻⁵ neutrophils with phorbol myristate acetate (PMA)—a neutrophil activator—and fMLP resulted in a high degree of CD16b shedding. Chemoattractant leukotreine B-4 (LTB-4) appears to have no effect on sCD16b concentration.

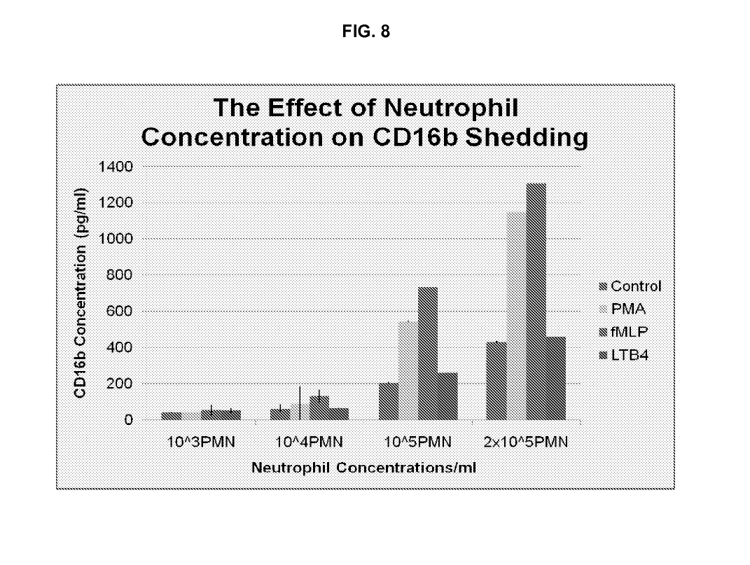

FIG. 9 illustrates the effects of Phospholipase C, an enzyme that cleaves GPI linked proteins, on CD16b shedding; Effects of PLC on sCD16b. Indicated concentrations of PLC were added to an eppendorf tubes containing 10⁻⁶ freshly isolated human neutrophils and incubated for 30 mins. Neutrophils were pelleted and the supernatant was collected and sCD16b concentration was measured using the ELISA assay.

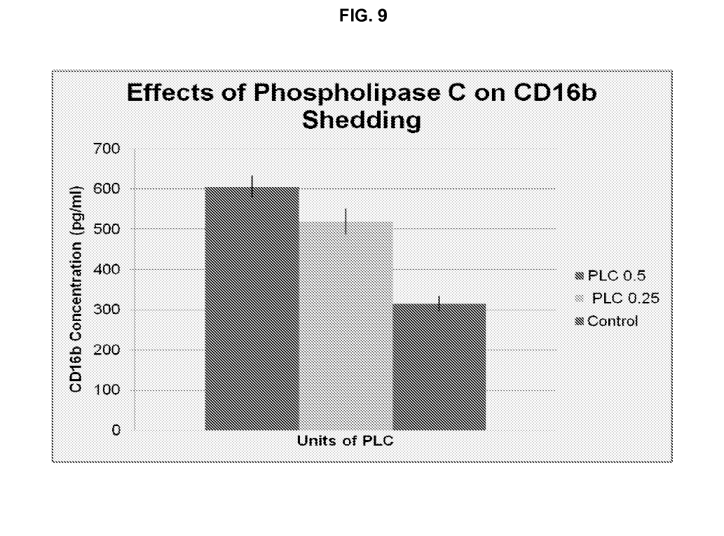

FIG. 10 illustrates the killing of bacteria by control and CD16b depleted neutrophils; Bactericidal Activity of PLC-treated and Untreated Neutrophils. Freshly isolated human neutrophils was incubated in 0.5 units of PLC for 30 min. Neutrophils were pelleted and re-suspended in PBS-GHSA buffer 10⁻⁷ S epidermidis were opsomized in 40% human serum for 30 minutes and then incubated with 4×10⁻⁵ neutrophils for 90 minutes and number of viable bacteria was assessed using a clonogenic assay. Shedding CD16b from human neutrophils reduces their capacity to kill bacteria in suspension.

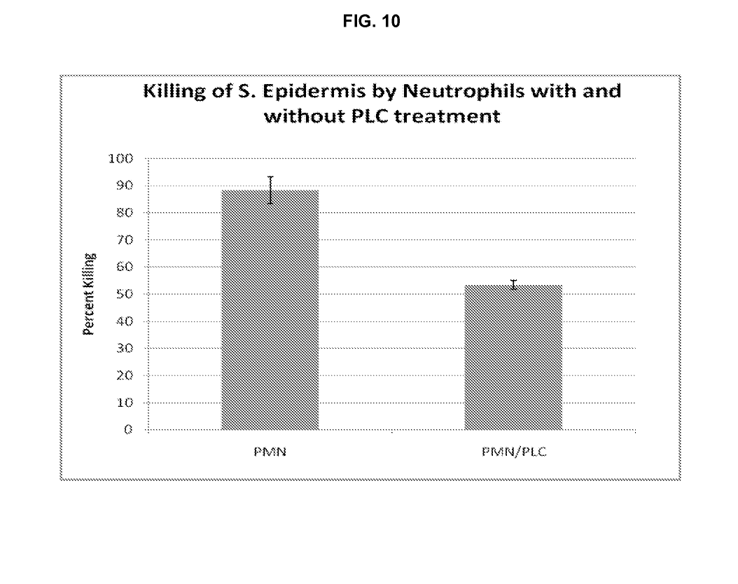

FIGS. 11A and 11B illustrate that shedding of CD16b reduces neutrophil chemotaxis through fibrin gels in response to LTB-4. FIG. 11A depicts the chemotaxis assay and 11B shows the results. Specifically, FIG. 11B shows the effect of CD16b Cleavage on Neutrophil Chemotaxis Through Fibris Gels. A chemotaxis chamber (8 micron pores) was placed on top of each assayed well of a 48-well plate as shown in FIG. 11A. The chambers were coated with 5 μl of thrombin (0.02 units/μL) and 100 μL of fibrogen (10 μL/mL) and the plate was incubated at 37° C. for 15 min. Fibrinogen polymerization into fibrin was terminated by the addition of 10 μL of PPACK 10⁻⁵ M) to each chamber, 10⁶ neutrophils pretreated (or untreated) with PLC for 30 minutes were added on top of the gel. The bottom compartment contained no chemoattractant (PBS-GHSA buffer) or 10⁻⁷ M LTB4. Neutrophils were allowed to migrate through the gel for six hours and migration into the lower compartment was determined using a Coulter Counter.

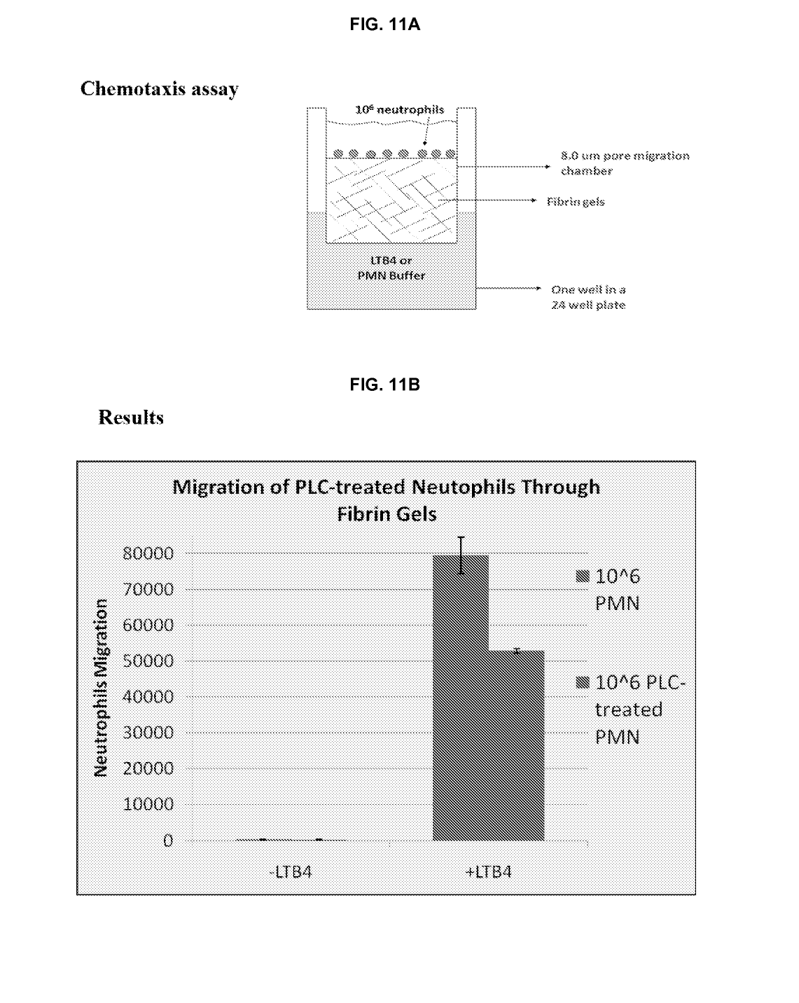

### ABBREVIATIONS AND DEFINITIONS

It is to be understood that the present invention is not limited to particular methods, reagents, compounds, compositions or biological systems, which can, of course, vary. It should also be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” or “a protein” includes a combination of two or more cells or two or more proteins, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used:

As used herein, the term “biological substance” and its grammatical equivalents, includes cells and their extra-cellular and intra-cellular constituents. For example, biological substances include pathogens, metabolites, DNA, RNA, lipids, proteins, carbohydrates, receptors, enzymes, hormones, growth factors, growth inhibitory factors, cells, organs, tissues, portions of cells, tissues, or organs, subcellular organelles, chemically reactive molecules like H⁺, superoxides, ATP, citric acid, protein albumin, as well as combinations or aggregate representations of these types of biological variables. In particular embodiments, the biological substance is a protein.

As used herein, the term “diagnosis” and its grammatical equivalents, means the testing of subjects to determine if they have a particular trait for use in a clinical decision. Diagnosis includes testing of subjects at risk of developing a particular disease resulting from infection by an infectious organism or a non infectious disease, such as an immunological disease or disorder. Diagnosis also includes testing of subjects who have developed particular symptoms to determine the cause of the symptoms. Diagnosis also includes prognosis, monitoring progress of a disease, and monitoring the efficacy of therapeutic regimens. The result of a diagnosis can be used to classify patients into groups for performance of clinical trials for administration of certain therapies.

As used herein, the term “nucleic acid” refers to deoxyribonucleotides, deoxyribonucleosides, ribonucleosides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. The term also refers to synthetically generated nucleic acid.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers. As used herein, the terms encompass amino acid chains of any length, including full length proteins (i.e., antigens), wherein the amino acid residues are linked by covalent peptide bonds. The term also refers to synthetically generated polypeptide, peptide or protein.

“Subject”, “mammalian subject” or “patient” refers to any mammalian patient or subject to which the methods and techniques described herein may be applied. “Mammal” or “mammalian” refers to human patients and non-human primates, as well as experimental animals such as rabbits, rats, and mice, and other animals. In addition to the methods and techniques described herein, conventional screening methods may be employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other methods, optionally coupled with the methods and techniques described herein, allow the clinician to select patients in need of therapy using the methods and formulations of the invention.

As used herein, the term “treating” and its grammatical equivalents include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

### DETAILED DESCRIPTION OF THE INVENTION

Among various aspects, the present invention is directed to methods of analyzing samples from a patient for the assessment of the immunological status of the patient. The processes generally involve the detection of particular biological substances in patient samples and correlating the levels of such substances to a susceptibility for various immunological diseases and disorders. In particular, blood, plasma, or serum may be collected and analyzed for the presence of certain biological substances, or markers, that are indicative of a compromised immune system. The results of this analysis are then suitable for use in diagnosis, prognosis, and determination of suitability of therapeutic interventions or modifications of existing treatment regimes. In particular embodiments, the methods and techniques are directed to predicting the onset of, or susceptibility to, sepsis in a neutropenic patient.

Neutrophils are the principal white blood cell involved in the defense against the bacteria that penetrate the skin and mucous membranes each day. They are short lived cells (living about 24-36 hours in blood), produced by radiation-sensitive stem cells in the bone marrow. They emigrate from the capillaries into the tissues in response to inflammatory signals initiated by invading bacteria. Clinicians have long recognized that individuals with less than a critical concentration of neutrophils in the blood are in danger of sepsis, but did not understand the reason for this threshold. The present inventors have disproved the conventional dogma that it is the concentration of neutrophils that determines the ability of these cells to control bacterial growth. Our published data shows that it is a Critical Neutrophil Concentration (CNC) in blood and tissues that controls bacteria that gain access to these sites (1,2).

However, not all neutropenic individuals will become septic. By way of background, neutrophils are produced in the bone marrow and are transported through the blood to sites of infection in the tissues. When they reach these sites they crawl out of capillaries and into tissue spaces where they ingest and kill bacteria. Thus, it is the tissue concentration of neutrophils that determines whether bacteria that have penetrated the skin or mucous membranes grow or are killed. The blood concentration, for example, provides only a rough estimate of the rate neutrophils enter and/or leave the blood. Thus, the critical information needed to determine whether a neutropenic patient is in danger of sepsis is the neutrophil concentration in tissues.

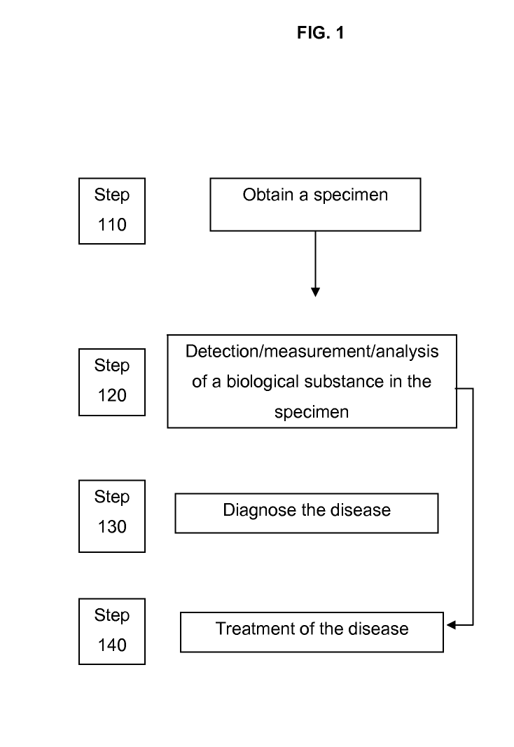

The methods of the present invention disclosed herein include methods for detecting, diagnosing, and treating a disease in a subject, by analyzing one or more biological substances in a patient sample. The steps of the methods of the present invention are depicted in FIG. 1. Without limiting the scope of the present invention, the steps can be performed independent of each other or one after the other. One or more steps may be skipped in the methods of the present invention. A sample is collected from a subject at step 110. One or more biological substances in the specimen is detected, measured and/or analyzed at step 120 by detection techniques described herein (e.g., ELISA). By way of example only some of the detection techniques are disclosed herein. A disease or susceptibility to a disease is diagnosed at step 130 based on the detection, measurement and/or analysis of the biological substance. A decision regarding treatment of the disease is made at step 140, the treatment decision being made based on the diagnosis.

The identification of the biological substances may involve one or more comparisons with reference specimens. The reference specimen may be obtained from the same subject or from a different subject who is either not affected with the disease or is a patient. The reference specimen could be obtained from one subject, multiple subjects or be synthetically generated. The identification may also involve the comparison of the identification data with the databases to identify the biological substance.

The steps of the methods of the present invention are generally provided herein. Without limiting the scope of the present invention, other techniques for collection of sample, detection of the biological substances and diagnosis of the disease are known in the art and are within the scope of the present invention.

Certain aspects of the present invention are directed to assays to measure the concentration of particular proteins which are predictive of, or indicative of an increased susceptibility to, an immunological disease (e.g., sepsis). In one particular embodiment, the protein is a neutrophil surface protein. Typically, the neutrophil surface protein is a shed protein; that is, it is formed by the proteolysis of ectodomains of membrane proteins. In a particularly preferred embodiment, the neutrophil surface protein is soluble CD16 b (also known as Fcγ receptor type IIIb (FcγRIIIb)).

In one embodiment, the present invention is directed to a rapid, sensitive, immuno-assay (e.g., ELISA) to measure the concentration of a neutrophil surface protein (e.g., soluble CD16b (sCD16b)) in a patient sample (e.g., blood, plasma, or other bodily fluid) for use in screening individuals exposed to sub-lethal doses of radiation. For instance, an enzyme linked immunosorbent assay (ELISA) kit is provided for the rapid detection of a neutrophil surface protein (e.g., sCD16b) which can be used on a routine basis in a clinical laboratory, and which allows a physician to: a) detect the presence of such proteins in a biological sample of a patient (e.g., blood, serum, plasma, urine, saliva, and seminal matter) and b) to predict susceptibility or onset of an immunological disease. In a preferred embodiment, the patient is a neutropenic patient, that is, the patient has a relatively low, or abnormally low number of neutrophils in the blood) and the immunological disease is sepsis.

Sample Collection

The biological sample or medium is preferably a biological fluid which can be obtained from said mammal, preferably a human patient. Such biological fluid could be a cellular biological fluid or an acellular biological fluid. Said biological fluid could be venous and capillary blood serum or plasma, seminal fluid, broncho-alveolar fluid, pleural fluid, sputum, nasal fluid, ascites fluids, synovial fluid, gastric bowel and faecal derivate samples or cerebrospinal fluid. In a particular embodiment, the biological sample is selected from blood, serum, plasma, seminal matter, and saliva.

In the sample collection step, specimens from patient are collected for analysis. Depending on the particular sample or specimen being collected, various collection methods may be employed. Where the sample to be collected is blood or a blood component, for example, one of the most common blood collection techniques, and perhaps the most well-known, is the manual collection of whole blood. As commonly understood, manual collection refers to a collection method where whole blood is allowed to drain from the patient and into a collection container without the use of external pumps or similar devices. Alternatively, so-called automated procedures may be employed, where blood is withdrawn from a patient and further processed by an instrument that typically includes a processing or separation device and pumps for moving blood or blood components into and out of the device. Regardless of whether the blood collection technique is manual or automated, withdrawing blood from the patient typically includes inserting a vein access device, such as a needle, into the donor's arm (and, more specifically, the donor's vein) and withdrawing blood from the donor through the needle. The “venipuncture” needle typically has attached to it, one end of a plastic tube that provides a flow path for the blood. The other end of the plastic tube terminates in one or more pre-attached plastic blood containers or bags for collecting the blood. The needle, tubing and containers make up a blood processing set which is pre-sterilized and disposed of after a single use. Alternative blood collection techniques include pricking the finger or other part of the patient and thereafter collecting the blood.

Suitable sample collection devices are well known to those skilled in the art. Preferably, a sample collection device can be a swab, a wooden spatula, bibulous materials such as a cotton ball, filter, or gauze pad, an absorbent-tipped applicator, capillary tube, a pipette, a needle or other piercing device, optionally coupled with a sampling tube and/or container.

In some instances, samples may be collected from individuals repeatedly over a longitudinal period of time (e.g., once a day, once a week, once a month, biannually or annually). Obtaining numerous samples from an individual over a period of time can be used to verify results from earlier detections and/or to identify an alteration as a result of, for example, drug treatment. Samples can be obtained from humans or non-humans. Preferably, samples are obtained from humans.

Detection and Analysis

In the present invention, a specimen is collected and analyzed using one or more analytical techniques including enzymatic techniques, immunological techniques (e.g., ELISA), fluorometric techniques, mass spectrography, HPLC, GLC, PCR, and other similar techniques. In one particular embodiment, the specimen is analyzed by an immunoassay (e.g., ELISA).

Immunoassays

Preferred embodiments of the invention include immunoassay for a detection and/or analysis of the biological substance. In immunoblotting, like the western blot of electrophoretically separated proteins, a single protein can be identified by its antibody. Suitable immunoassays include competitive binding immunoassays where analyte competes with a labeled antigen for a limited pool of antibody molecules (e.g., radioimmunoassay, EMIT). Other suitable immunoassays can be non-competitive where antibody is present in excess and is labeled. As analyte antigen complex is increased, the amount of labeled antibody-antigen complex may also increase (e.g., ELISA). Antibodies can be polyclonal if produced by antigen injection into an experimental animal, or monoclonal if produced by cell fusion and cell culture techniques. In many immunoassays, the antibody may serve as a specific reagent for the analyte antigen.

Without limiting the scope and content of the present invention, some of the types of immunoassays are, by way of example only, RIAs (radioimmunoassay), enzyme immunoassays like ELISA (enzyme-linked immunosorbent assay), EMIT (enzyme multiplied immunoassay technique), microparticle enzyme immunoassay (META), LIA (luminescent immunoassay), and FIA (fluorescent immunoassay). These techniques can be used to detect biological substances in accordance with the invention. The antibodies, whether used as primary or secondary antibodies, may also be labeled with radioisotopes (e.g., ¹²⁵I), fluorescent dyes (e.g., FITC) or enzymes (e.g., HRP or AP) which may catalyse fluorogenic or luminogenic reactions. Several immunoassays are described in further detail below.

EMIT (Enzyme Multiplied Immunoassay Technique): EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein.

ELISA (Enzyme Linked Immunosorbent Assay): Certain preferred embodiments of the invention include ELISA to detect the biological substances. In general, ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test may require the antibodies to be fixed to a solid surface, such as the inner surface of a test tube, and a preparation of the same antibodies coupled to an enzyme. The enzyme may be one (e.g., β-galactosidase) that produces a colored product from a colorless substrate. The test, for example, may be performed by filling the tube with the antigen solution (e.g., protein) to be assayed. Any antigen molecules present may bind to the immobilized antibody molecules. The antibody-enzyme conjugate may be added to the reaction mixture. The antibody part of the conjugate binds to any antigen molecules that were bound previously, creating an antibody-antigen-antibody “sandwich”. After washing away any unbound conjugate, the substrate solution may be added. After a set interval, the reaction is stopped (e.g., by adding 1 N NaOH) and the concentration of colored product formed is measured in a spectrophotometer. The intensity of color is proportional to the concentration of bound antigen.

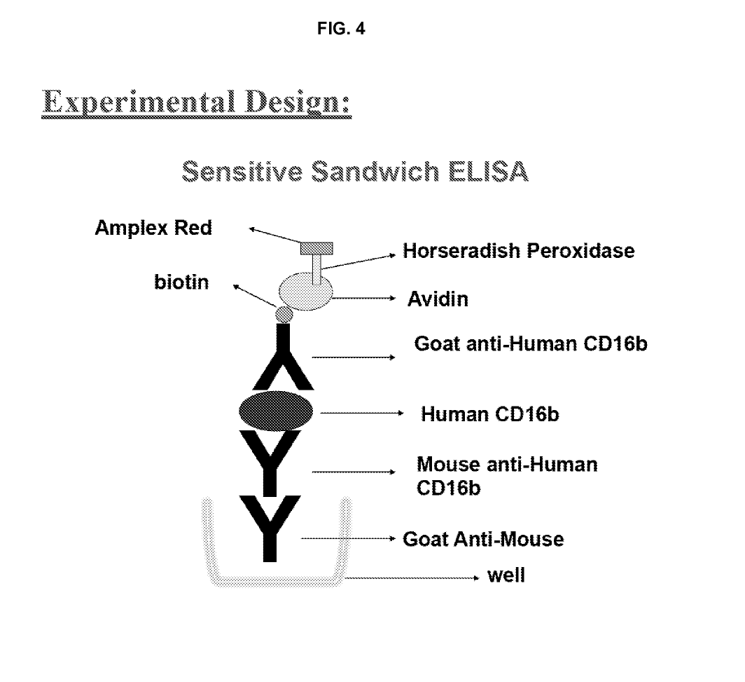

In one exemplary ELISA, antibodies of the neutrophil surface protein(s) are immobilized onto a selected surface exhibiting protein affinity, such as a well in a polystyrene microtiter plate. Then, a sample from a patient is added to the wells. After binding and/or washing to remove non-specifically bound immune complexes, the bound therapeutic antibody:antigen complex may be detected. Detection is generally achieved by the addition of second antibody that is linked to a detectable label. This type of ELISA can be referred to as a “sandwich ELISA”. See FIG. 4.

The antibodies used in the methods of the invention may be monospecific, bispecific, trispecific or of even greater multispecificity. In addition the antibodies may be monovalent, bivalent, trivalent or of even greater multivalency. Furthermore, the antibodies of the invention may be from any animal origin including, but not limited to, birds and mammals. In specific embodiments, the antibodies are human, murine, rat, sheep, rabbit, goat, guinea pig, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins.

The antibodies used in the present invention may be described or specified in terms of the epitope(s) or portion(s) of a polypeptide to which they recognize or specifically bind. Or, the antibodies may be described based upon their ability to bind to specific conformations of the antigen, or specific modification (e.g., cleavage or chemical, natural or otherwise, modification of sequence).

The specificity of the antibodies used in present invention may also be described or specified in terms of their binding affinity towards the antigen (epitope) or of by their cross-reactivity. Specific examples of binding affinities encompassed in the present invention include but are not limited to those with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷, 5×10⁻⁸ M, 10⁻⁸ , 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. As used herein, a substantially equivalent binding affinity means within the same order of magnitude of the dissociation constant.

The antibodies used in the invention also include derivatives that are modified, for example, by covalent attachment of any type of molecule to the antibody such that covalent attachment does not prevent the antibody from generating an anti-idiotypic response. Examples of modifications to antibodies include but are not limited to, glycosylation, acetylation, pegylation, phosphorylation, amidation, derivatization by known protecting/blocking groups, proteolytic cleavage, linkage to a cellular ligand or other composition, such as a signaling moiety, a label etc. In addition, the antibodies may be linked or attached to solid substrates, such as, but not limited to, beads, particles, glass surfaces, plastic surfaces, ceramic surfaces, metal surfaces. Any of numerous chemical modifications may be carried out by known techniques, including, but not limited to, specific chemical cleavage, acetylation, biotinylation, farnesylation, formylation, inhibition of glycosylation by tunicamycin and the like. Additionally, the derivative may contain one or more non-classical or synthetic amino acids.

The antibodies used in the present invention may be generated by any suitable method known in the art. Polyclonal antibodies can be produced by various procedures well known in the art. For example, an antigen or an epitope on the antigen can be administered to various host animals including, but not limited to, rabbits, goats, chickens, mice, rats, to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (both of which are incorporated by reference).

Methods for producing and screening for specific antibodies using hybridoma technology are routine and well known in the art such as, but not limited to, immunizing a mouse, hamster, or rat. Additionally, newer methods to produce rabbit and other mammalian monoclonal antibodies may be available to produce and screen for antibodies. Once an immune response is detected, the mouse spleen is harvested and splenocytes isolated. The splenocytes are then fused by well known techniques to any suitable myeloma cells, for example cells from cell line SP2/0 available from the ATCC. Hybridomas are selected and cloned by limited dilution. The hybridoma clones can then be assayed by methods known in the art for cells that secrete antibodies capable of binding a biomarker of the present invention. Ascites fluid, which generally contains high levels of antibodies, can be generated by immunizing mice with positive hybridoma clones. In addition, antibodies can be produced using a variety of alternate methods, including but not limited to bioreactors and standard tissue culture methods, to name a few.

The antibodies used the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library. Phage expressing an antigen binding domain that binds the antigen of interest can be selected or identified with the antigen of interest, such as using a labeled antigen or antigen bound or captured to a solid surface or bead. The phage used in these methods are typically filamentous phage including, but not limited to, fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, all of which are incorporated by reference.

Antibody fragments which recognize specific epitopes may be generated by known techniques. For example, Fab and F(ab)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

ELISA can also be adapted to measure the concentration of antibodies, in which case, the wells are coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced is proportional to the amount of enzyme-labeled antibodies bound (and thus to the concentration of the antibodies being assayed).

Radioimmunoassay: Some embodiments of the invention employ radioimmunoassays to detect the biological substance in the specimen. Radioactive isotopes can be used to study in vivo metabolism, distribution, and binding of small amount of compounds. Radioactive isotopes of ¹H, ¹²C, ³¹P, ³²S, and ¹²⁷I are commonly used, such as ³H, ¹⁴C, ³²P, ³⁵S, and ¹²⁵I.

In receptor fixation methods in 96 well plates, receptors may be fixed in each well by using antibody or chemical methods and radioactive labeled ligands may be added to each well to induce binding. Unbound ligands may be washed out and then the standard can be determined by quantitative analysis of radioactivity of bound ligands or that of washed-out ligands. Then, addition of screening target compounds may induce competitive binding reaction with receptors. If the compounds show higher affinity to receptors than standard radioactive ligands, most of radioactive ligands would not bind to receptors and may be left in solution. Therefore, by analyzing quantity of bound radioactive ligands (or washed-out ligands), testing compounds' affinity to receptors can be indicated.

In certain embodiments, the filter membrane method may be employed when receptors cannot be fixed to 96 well plates or when ligand binding is preferably performed in solution phase. In other words, after ligand-receptor binding reaction in solution, if the reaction solution is filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Fluorescence Immunoassays: Other embodiments of the invention include fluorescence immunoassays for a detection of the biological substance. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogenous or homogenous. Heterogenous immunoassays comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody is used). Homogenous immunoassays comprise no physical separation. Double-antibody fluorophore-labeled antigen participates in an equilibrium reaction with antibodies directed against both the antigen and the fluorophore. Labeled and unlabeled antigen may compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that selectively measures fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody is adhered to a solid phase and a scanning probe microscope is utilized to detect antigen/antibody complexes which may be present on the surface of the solid phase. The use of scanning tunneling microscopy eliminates the need for labels which normally is utilized in many immunoassay systems to detect antigen/antibody complexes.

Other suitable analytical tests which may be used to detect the biological substance of interest are described in further detail below.

Polymerase Chain Reaction (PCR)

In general, the polymerase chain reaction (PCR) is a process for amplifying one or more desired specific nucleic acid sequences found in a nucleic acid. Because large amounts of a specific sequence may be produced by this process, it is used for improving the efficiency of cloning DNA or messenger RNA and for amplifying a target sequence to facilitate detection thereof. PCR involves a chain reaction for producing, in exponential quantities relative to the number of reaction steps involved, at least one specific nucleic acid sequence given (a) that the ends of the required sequence are known in sufficient detail that oligonucleotides can be synthesized which will hybridize to them, and (b) that a small amount of the sequence is available to initiate the chain reaction. The product of the chain reaction would be a discrete nucleic acid duplex with termini corresponding to the ends of the specific primers employed.

Any source of nucleic acid, in purified or non purified form, can be utilized as the starting nucleic acid or acids, provided it contains or is suspected of containing the specific nucleic acid sequence desired. Thus, the process may employ, for example, DNA or RNA, including messenger RNA, which DNA or RNA may be single stranded or double stranded. In addition, a DNA-RNA hybrid which contains one strand of each may be utilized. A mixture of any of these nucleic acids may also be employed, or the nucleic acid produced from a previous amplification reaction herein using the same or different primers may be so utilized. The specific nucleic acid sequence to be amplified may be only a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as a portion of the β-globin gene contained in whole human DNA or a portion of nucleic acid sequence due to a particular microorganism which organism might constitute only a very minor fraction of a particular biological sample. The starting nucleic acid may contain more than one desired specific nucleic acid sequence which may be the same or different. Therefore, it is useful not only for producing large amounts of one specific nucleic acid sequence, but also for amplifying simultaneously more than one different specific nucleic acid sequence located on the same or different nucleic acid molecules.

The nucleic acid or acids may be obtained from any source, for example, from plasmids such as pBR322, from cloned DNA or RNA, or from natural DNA or RNA from any source, including bacteria, yeast, viruses, and higher organisms such as plants or animals. In a particular embodiment, DNA or RNA may be extracted from blood, plasma, serum, or other bodily fluids and/or tissue material (e.g., cells).

It will be understood that the primer described and used hereinafter may refer to more than one primer, particularly in the case where there is some ambiguity in the information regarding the terminal sequence(s) of the fragment to be amplified. For instance, in the case where a nucleic acid sequence is inferred from protein sequence information a collection of primers containing sequences representing all possible codon variations based on degeneracy of the genetic code will be used for each strand. One primer from this collection will be 100% homologous with the end of the desired sequence to be amplified. It will also be understood that an appropriate agent may be added for inducing or catalyzing the primer extension reaction and that the reaction is allowed to occur under conditions known in the art. The inducing agent may be any compound or system which will function to accomplish the synthesis of primer extension products, including enzymes. Suitable enzymes for this purpose may include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis can be initiated at the 3′ end of each primer and proceed in the 5′ direction along the template strand, until synthesis terminates, producing molecules of different lengths. There may be inducing agents, however, which initiate synthesis at the 5′ end and proceed in the other direction, using the same process as described above.

The newly synthesized strand and its complementary nucleic acid strand form a double-stranded molecule which can be used in the succeeding steps of the process. In the next step, the strands of the double-stranded molecule may be separated to provide single-stranded molecules. New nucleic acid may be synthesized on the single-stranded molecules. Additional inducing agent, nucleotides and primers may be added if necessary for the reaction to proceed under the conditions prescribed above. Again, the synthesis would be initiated at one end of the oligonucleotide primers and would proceed along the single strands of the template to produce additional nucleic acid. After this step, half of the extension product would consist of the specific nucleic acid sequence bounded by the two primers. The steps of strand separation and extension product synthesis can be repeated as often as needed to produce the desired quantity of the specific nucleic acid sequence. The amount of the specific nucleic acid sequence produced would accumulate in an exponential fashion. After the appropriate length of time has passed to produce the desired amount of the specific nucleic acid sequence, the reaction may be halted by inactivating the enzymes in any known manner or separating the components of the reaction.

Amplification may be useful when the amount of nucleic acid available for analysis is very small. Amplification is particularly useful if such an analysis is to be done on a small sample using non-radioactive detection techniques which may be inherently insensitive, or where radioactive techniques are being employed but where rapid detection is desirable. Any known techniques for nucleic acid (e.g., DNA and RNA) amplification can be used with the assays described herein. Preferred amplification techniques are the polymerase chain reaction (PCR) methodologies which comprise solution PCR and in situ PCR. The analysis is not limited to the use of straightforward PCR. A system of nested primers may be used for example. Other suitable amplification methods known in the field can also be applied including, but not limited to, ligase chain reaction (LCR), strand displacement amplification (SDA), self-sustained sequence replication (3SR), array based test, and TAQMAN.

In general, amplification may refer to any in vitro method for increasing the number of copies of a nucleic acid sequence with the use of a DNA polymerase. Nucleic acid amplification results in the incorporation of nucleotides into a DNA molecule or primer thereby forming a new DNA molecule complementary to a DNA template. The newly formed DNA molecule and its template can be used as templates to synthesize additional DNA molecules. For instance, one amplification reaction may consist of many rounds of DNA replication. DNA amplification reactions include, for example, polymerase chain reactions (PCR). One PCR reaction may consist of 5-100 “cycles” of denaturation, annealing, and synthesis of a DNA molecule.

Fluorescence Microscopy

Some embodiments of the invention include fluorescence microscopy for a detection of the biological substance. Fluorescence microscopy enables the molecular composition of the structures being observed to be identified through the use of fluorescently-labeled probes of high chemical specificity such as antibodies. It can be done by directly conjugating a fluorophore to a protein and introducing this back into a cell. Fluorescent analogue may behave like the native protein and can therefore serve to reveal the distribution and behavior of this protein in the cell. Along with NMR, infrared spectroscopy, circular dichroism and other techniques, protein intrinsic fluorescence decay and its associated observation of fluorescence anisotropy, collisional quenching and resonance energy transfer are techniques for protein detection.

Various naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry. Without limiting the scope of the present invention, some of the probes are as following:

Labels: Sensitivity and safety (compared to radioactive methods) of fluorescence has led to an increasing use for specific labeling of nucleic acids, proteins and other biomolecules. Besides fluorescein, other fluorescent labels cover the whole range from 400 to 820 nm. By way of example only, some of the labels are, fluorescein and its derivatives, carboxyfluoresceins, rhodamines and their derivatives, atto labels, fluorescent red and fluorescent orange: Cy3/Cy5 alternatives, lanthanide complexes with long lifetimes, long wavelength labels—up to 800 nm, DY cyanine labels, and phycobili proteins.

Conjugates: Antibody conjugates can be generated with specificity for virtually any epitope and are therefore, applicable to imaging a wide range of biomolecules. By way of example only, some of the conjugates are, isothiocyanate conjugates, streptavidin conjugates, and biotin conjugates.

Enzyme Substrates: By way of example only, some of the enzyme substrates are fluorogenic and chromogenic substrates.

Micro- and Nanoparticles: By way of example only, some of the fluorochromes are: FITC (green fluorescence, excitation/emission=506/529 nm), rhodamine B (orange fluorescence, excitation/emission=560/584 nm), and nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc.

Molecular Rotors: Fluorescent molecular rotors are sensors of microenvironmental restriction that become fluorescent when their rotation is constrained. Few examples of molecular constraint include increased dye (aggregation), binding to antibodies, or being trapped in the polymerization of actin.

IEF-Markers: IEF (isoelectric focusing) is an analytical tool for the separation of ampholytes, mainly proteins. An advantage for IEF-Gel electrophoresis with fluorescent IEF-marker is the possibility to directly observe the formation of gradient. Fluorescent IEF-marker can also be detected by UV-absorption at 280 nm (20° C.).

Any or all of these fluorescent probes can be used for the detection of the biological substances of interest in the method of the invention. A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used. A FACS machine originally runs cells through a capillary tube and separate cells by detecting their fluorescent intensities.

Nuclear Magnetic Resonance (NMR)

Some embodiments of the invention include NMR for detection of a biological substance. NMR spectroscopy is capable of determining the structures of biological macromolecules like proteins and nucleic acids at atomic resolution. In addition, it is possible to study time dependent phenomena with NMR, such as intramolecular dynamics in macromolecules, reaction kinetics, molecular recognition or protein folding. Heteronuclei like ¹⁵N, ¹³C, and ²H, can be incorporated in proteins by uniform or selective isotopic labeling. Additionally, some new information about structure and dynamics of macromolecules can be determined with these methods.

X-Ray Crystallography

Some embodiments of the invention include X-ray crystallography for detection of the biological substance. X-ray crystallography is a technique in which the pattern produced by the diffraction of X-rays through the closely spaced lattice of atoms in a crystal is recorded and then analyzed to reveal the nature of that lattice. This generally leads to an understanding of the material and molecular structure of a substance. The spacings in the crystal lattice can be determined using Bragg's law. X-ray diffraction is commonly carried out using single crystals of a material, but if these are not available, microcrystalline powdered samples may also be used which may require different equipment.

Fluorescence Spectroscopy

Some embodiments of the invention include fluorescence spectroscopy for detection of the biological substance. By way of example only, conventional fluorometry is measurement of emission light intensities at defined wavelengths for a certain emission maxima of a fluorophore. Total fluorometry is a collection of data for a continuum of absorption as well as emission wavelengths. Fluorescence polarization is when polarized light is used for excitation and binding of fluorochrome-labeled antigens to specific antibodies. Line narrowing spectroscopy is low-temperature solid-state spectroscopy that derives its selectivity from the narrow-line emission spectra.

Time-dependent fluorescence spectroscopy comprises time-resolved measurements containing more information than steady-state measurements, since the steady-state values represent the time average of time-resolved determinations. It is a single photon timing technique where the time between an excitation light pulse and the first photon emitted by the sample is measured.

Matrix Assisted Laser Desorption Ionization Time-of-Flight Mass Spectrometry (MALDI TOF-MS)

Some embodiments of the invention include MALDI TOF-MS for detection of a biological substance in a patient specimen. MALDI TOF-MS provides accurate mass determinations and primary sequence information. Improved mass resolution in MALDI TOF-MS can be obtained by the utilization of a single-stage or a dual-stage reflectron (RETOF-MS). In the reflectron mass spectrum, the isotopic multiplet is well resolved producing a full width half maximum (FWHM) mass resolution of about 3400. Mass resolutions up to 6000 (FWHM) can be obtained for peptides up to about 3000 Da with RETOF-MS. Enhancing the mass resolution can also increase the mass accuracy when determining the ion's mass.

Both linear and reflectron MALDI-TOF-MS can be utilized for molecular weight determinations of molecular ions and enzymatic digests leading to structural information of proteins. These digests are typically mass analyzed with or without purification prior to molecular weight determinations. Varieties of methodologies have been developed to obtain primary sequence information for proteins and peptides utilizing MALDI TOF-MS. In general, two different approaches can be taken. The first method is known as protein ladder sequencing and can be employed to produce structurally informative fragments of the analyte prior to insertion into the TOF mass spectrometer and subsequent analysis. The second approach utilizes the phenomenon of metastable ion decay that occurs inside the TOF mass spectrometer to produce sequence information.

The ladder sequencing with TOF-MS consists of either a time-dependent or concentration-dependent chemical degradation from either the N- or C-terminus of the protein/peptide into fragments, each of which differs by one amino acid residue. The mixture is mass analyzed in a single MALDI-TOF-MS experiment with mass differences between adjacent mass spectral peaks corresponding to a specific amino acid residue. The order of occurrence in the mass spectrum defines the sequence of amino acids in the original protein/peptide.

Post-source decay with RETOF-MS MALDI is an ionization technique that produces intact protonated pseudomolecular ion species. A significant degree of metastable ion decay occurs after ion acceleration and prior to detection. The ion fragments produced from the metastable ion decay of peptides and proteins typically include both neutral molecule losses (such as water, ammonia and portions of the amino acid side chains) and random cleavage at peptide bonds. In-source decay with linear TOF-MS is an alternative approach to RETOF-MS for studying metastable ion decay of MALDI generated ions. Primary structural information for peptides and proteins can be obtained by this method. Coherent mass spectral peaks can be produced from these metastable decayed ions giving rise to significant structural information for peptides and proteins.

Surface-Enhanced Laser Desorption Ionization-Time of Flight (SELDI-TOF)

Some embodiments of the invention include SELDI TOF-MS for detection of a biological substance. This technique utilizes stainless steel or aluminum-based supports, or chips, engineered with chemical (hydrophilic, hydrophobic, pre-activated, normal-phase, immobilized metal affinity, and cationic or anionic) or biological (antibody, antigen binding fragments (e.g. scFv), DNA, enzyme, or receptor) bait surfaces of 1-2 mm in diameter. These varied chemical and biochemical surfaces allow differential capture of proteins based on the intrinsic properties of the proteins themselves. Solubilized tissue or body fluids in volumes as small as 0.1 μl can be directly applied to these surfaces, where proteins with affinities to the bait surface may bind. Following a series of washes to remove non-specifically or weakly bound proteins, the bound proteins are laser desorbed and ionized for MS analysis. Masses of proteins ranging from small peptides of less than 1000 Da up to proteins of greater than 300 kDa can be calculated based on time-of-flight. As mixtures of proteins may be analyzed within different samples, a unique sample fingerprint or signature may result for each sample tested. Consequently, patterns of masses rather than actual protein identifications can be produced by SELDI analysis. These mass spectral patterns can be used to differentiate patient samples from one another, such as diseased from normal.

UV-VIS

Some embodiments of the invention include optical absorption spectroscopy (UV/VIS) for detection of a biological substance. UV/VIS provides light absorption data which helps in the determination of concentration of macromolecules such as, proteins, DNA, nucleotides etc. Organic dyes can be used to enhance the absorption and to shift the absorption into the visible range (e.g., coomassie blue reagents). Resonance raman spectroscopy (RRS) can be used to study molecular structure and dynamics. RRS helps in investigating specific parts of macromolecules by using different excitation wavelengths.

Liquid Chromatography (LC)

Some embodiments of the invention include LC for a detection of biological substance. Examples of LC are but not limited to, affinity chromatography, gel filtration chromatography, anion exchange chromatography, cation exchange chromatography, diode array-LC and high performance liquid chromatography (HPLC).

Gel filtration chromatography separates proteins, peptides, and oligonucleotides on the basis of size. Molecules may move through a bed of porous beads, diffusing into the beads to greater or lesser degrees. Smaller molecules may diffuse further into the pores of the beads and therefore move through the bed more slowly, while larger molecules may enter less or not at all and thus move through the bed more quickly. Both molecular weight and three dimensional shapes contribute to the degree of retention. Gel Filtration Chromatography may be used for analysis of molecular size, for separations of components in a mixture, or for salt removal or buffer exchange from a preparation of macromolecules.

Affinity chromatography is the process of bioselective adsorption and subsequent recovery of a compound from an immobilized ligand. This process allows for the specific and efficient purification of many diverse proteins and other compounds. Ion exchange chromatography separates molecules based on differences between the overall charges of the proteins. It can be used for the purification of protein, oligonucleotides, peptides, or other charged molecules.

HPLC can be used in the separation, purification and detection of biological substances from a specimen. Crude tissue extracts or bodily fluids may be loaded directly onto the HPLC system and mobilized by gradient elution. Rechromatography under the identical conditions is an option if further purification is warranted or necessary. Reversed phase chromatography (RPC) can be utilized in the process of protein structure determination. HPLC may be coupled with MS.

The size-exclusion chromatography (SEC) and ion-exchange chromatography (IEC) can be used for separation and purification of biologically active proteins, such as enzymes, hormones, and antibodies. In liquid affinity chromatography (LAC), interaction may be based on binding of the protein due to mimicry of substrate, receptor, etc. The protein may be eluted by introducing a competitive binding agent or altering the protein configuration which may facilitate dissociation. One suitable procedure that can be used in the separation of membrane proteins is the use of nonionic detergents, such as Triton X-100, or protein solubilization by organic solvents with IEC.

Diode array detector-liquid chromatography (DAD-LC) provides complete, multiple spectra for each HPLC peak, which, by comparison, can provide indication of peak purity. These data can also assign presence of tyr, trp, phe, and possibly others (his, met, cys) and can quantitate these amino acids by 2nd derivative or multi-component analysis. By a post-column derivatization, DAD-LC can also identify and quantitate cys, his and arg in individual peptides. Thus, it is possible to analyze for 6 of the 20 amino acids of each separated peptide in a single LC run, and information can be obtained about presence or absence of these amino acids in a given peptide in a single step. This is assisted by knowing the number of residues in each peptide.

Electrophoresis

Some embodiments of the invention include electrophoresis for detection of a biological substance. Electrophoresis can be gel electrophoresis or capillary electrophoresis.

Gel Electrophoresis: Gel electrophoresis is a technique that can be used for the separation of proteins. During electrophoresis, macromolecules are forced to move through pores when an electrical current is applied. Their rate of migration through the electric field depends on strength of the field, size and shape of the molecules, relative hydrophobicity of the samples, and on an ionic strength and temperature of a buffer in which the molecules are moving. After staining, the separated macromolecules in each lane can be seen in a series of bands spread from one end of the gel to the other. Using this technology it is possible to separate and identify protein molecules that differ by as little as a single amino acid. Also, gel electrophoresis allows determination of crucial properties of a protein such as its isoelectric point and approximate molecular weight. Electrofocusing or isoelectric focusing is a technique for separating different molecules by their electric charge differences (if they have any charge). It is a type of zone electrophoresis that takes advantage of the fact that a molecule's charge changes as the pH of its surroundings changes.

Capillary Electrophoresis: Capillary electrophoresis is a collection of a range of separation techniques which may involve the application of high voltages across buffer filled capillaries to achieve separations. The variations include separation based on size and charge differences between analytes (termed capillary zone electrophoresis (CZE) or free solution CE (FSCE)), separation of neutral compounds using surfactant micelles (micellar electrokinetic capillary chromatography (MECC) or sometimes referred to as MEKC) sieving of solutes through a gel network (capillary gel electrophoresis, GCE), separation of cations (or anions) based on electrophoretic mobility (capillary isotachophoresis, CITP), and separation of zwitterionic solutes within a pH gradient (capillary isoelectric focusing, LIEF). Capillary electrochromatography (CEC) can be an associated electrokinetic separation technique which involves applying voltages across capillaries filled with silica gel stationary phases. Separation selectivity in CEO can be a combination of both electrophoretic and chromatographic processes. Many of the CE separation techniques rely on the presence of an electrically induced flow of solution (electroosmotic flow, EOF) within the capillary to pump solutes towards the detector.

Arrays

Some embodiments of the invention include arrays for detection of a biological substance. Arrays involve performing parallel analysis of multiple samples against known protein targets. The development of various microarray platforms can enable and accelerate the determination of protein abundance, localization, and interactions in a cell, tissue, or fluid sample. Microarrays provide a platform that allows identification of protein interaction or function against a characterized set of proteins, antibodies, or peptides. Protein-based chips array proteins on a small surface and can directly measure the levels of proteins in tissues using fluorescence-based imaging. Proteins can be arrayed on either flat solid phases or in capillary systems (microfluidic arrays), and several different proteins can be applied to these arrays. In addition to the use of antibodies as array probes, single-stranded oligonucleotides, whose specificity is optimized by in vitro elution (aptamers), offer a viable alternative. Nonspecific protein stains can be then used to detect bound proteins.

Suitable arrays include, but are not limited to, bead arrays, bead based arrays, bioarrays, bioelectronic arrays, cDNA arrays, cell arrays, DNA arrays, gene arrays, gene expression arrays, frozen cell arrays, genome arrays, high density oligonucleotide arrays, hybridization arrays, microcantilever arrays, microelectronic arrays, multiplex DNA hybridization arrays, nanoarrays, oligonucleotide arrays, oligosaccharide arrays, planar arrays, protein arrays, solution arrays, spotted arrays, tissue arrays, exon arrays, filter arrays, macroarrays, small molecule microarrays, suspension arrays, theme arrays, tiling arrays, and transcript arrays.

Sensors

Some embodiments of the invention include sensors for detection of a biological substance. Sensors can be used for both in vivo and in vitro detection. Sensors can be chemical sensors, optical sensors, and biosensors. Chemical sensors are miniaturized analytical devices which may deliver real-time and online information on the presence of specific compounds or ions in complex samples. Optical sensors are based on measurement of either intrinsic optical properties of analytes, or of optical properties of indicator dyes or labeled biomolecules attached to solid supports. Biosensors can be affinity biosensor based on capabilities of enzymes to convert substrates into products or catalytic biosensors. Biosensors detect antibody and analyte complexes using a variety of physical methods. Some biosensors measure the change in surface charge that occurs when analyte is bound to antibodies or other binding agents, which in turn are bound to a surface. Other biosensors use binding agents attached to a surface and measure a change in a physical property of the support, other than surface charge, upon binding of analyte. Some biosensor techniques use a specific property of a labeled binding agent or antigen to produce a measurable change.

Methods for Identifying Proteins of Interest

Protein identification methods by way of example only include low-throughput sequencing through Edman degradation, mass spectrometry techniques, peptide mass fingerprinting, de novo sequencing, and antibody-based assays. The protein quantification assays include fluorescent dye gel staining, tagging or chemical modification methods (i.e. isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure include x-ray crystallography and NMR spectroscopy. Described below are a few of the methods for identifying proteins in the present invention.

Protein sequencing: N-terminal sequencing aids in the identification of unknown proteins, confirm recombinant protein identity and fidelity (reading frame, translation start point, etc.), aid the interpretation of NMR and crystallographic data, demonstrate degrees of identity between proteins, or provide data for the design of synthetic peptides for antibody generation, etc. N-terminal sequencing utilizes the Edman degradative chemistry, sequentially removing amino acid residues from the N-terminus of the protein and identifying them by reverse-phase HPLC. Sensitivity can be at the level of 100s femtomoles and long sequence reads (20-40 residues) can often be obtained from a few 10 s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g., using cyanogen bromide) may allow a mixture of amino acids to be generated in each cycle of the instrument, which can be subjected to database analysis in order to interpret meaningful sequence information. C-terminal sequencing is a post-translational modification, affecting the structure and activity of a protein. Various disease situations can be associated with impaired protein processing and C-terminal sequencing provides an additional tool for the investigation of protein structure and processing mechanisms.

Proteome analyses: Proteomics can be identified primarily by computer search algorithms that assign sequences to a set of empirically acquired mass/intensity data which are generated from conducting electrospray ionization (ESI), matrix-assisted laser desorption/ionization (MALDI-TOF), or three-dimensional quadrupole ion traps on the protein of interest.

Diagnosis

The identification and analysis of biological substances as disclosed herein has numerous therapeutic and diagnostic applications. Clinical applications include, for example, detection of disease, distinguishing disease states to inform prognosis, selection of therapy, and/or prediction of therapeutic response, disease staging, identification of disease processes, prediction of efficacy of therapy, monitoring of patients trajectories (e.g., prior to onset of disease), prediction of adverse response, monitoring of therapy associated with efficacy and toxicity, and detection of recurrence.

Measuring a concentration of the biological substance can aid in the diagnosis of a course of a disease. A biological substance, for example, growth factor, may be one that is specific for or predictive of the patient's specific disease. Alternatively, a panel of two or more specific or non-specific growth factors may be monitored. The concentrations of either an individual factor or several factors, in the biological sample of the patient may be affected by the disease.

The presence or increase or decrease of biological substances' concentration allows the physician or veterinarian to predict the course or onset of the disease or the efficacy of treatment regimes. If, for example, a patient who had a certain type of disease, which was treated, subsequently exhibits an increase in the concentration of biological substances that is associated with that or some other disease, whether related or unrelated to the original disease, the physician or veterinarian can predict that the patient may have progression of the disease in the future or predict a higher risk of fatality in the patient. In addition, the amount of biological substances may be predictive of the outcome of the patient, e.g., whether an immunological disorder such as sepsis may arise as a result of the patient's neutropenia.

The diagnosis of the disease as disclosed herein can be used to enable or assist in the pharmaceutical drug development process for therapeutic agents. The analysis can be used to diagnose disease for patients enrolling in a clinical trial. The diagnosis can indicate the state of the disease of patients undergoing treatment in clinical trials, and show changes in the state during the treatment. For instance, the diagnosis can demonstrate the efficacy of a treatment, and can be used to stratify patients according to their responses to various therapies.

The methods of the present invention can be used to evaluate the efficacy of treatments over time. By way of example, samples can be obtained from a patient over a period of time as the patient is undergoing treatment. The biological specimens from the different samples can be compared to each other to determine the efficacy of the treatment. Also, the methods described herein can be used to compare the efficacies of different therapies and/or responses to one or more treatments in different populations (e.g., different age groups, ethnicities, family histories, etc.).

Immunological Diseases

Without limiting the scope of the present invention, the examples of some of the diseases which can be predicted or diagnosed by detecting the biological substance, is provided herein. However, these examples are not intended to limit the scope of the invention. In general, the disease is an immunological disorder, though it is contemplated that other diseases may be detected or predicted using the methods and techniques described herein. This may include, for example, infections, hematological disorders, oncological disorders, endocronological disorders, metabolic disorders, neurological disorders, vascular disorders, mast cell disorders, psychiatric disorders, neoplastic disorders, nutritional disorders, post irradiation disorders, and changes in the trace metal metabolism.

Inflammatory disease states include systemic inflammatory conditions and conditions associated locally with migration and attraction of monocytes, leukocytes and/or neutrophils. Inflammation may result from infection with pathogenic organisms (including gram-positive bacteria, gram-negative bacteria, viruses, fungi, and parasites such as protozoa and helminths), transplant rejection (including rejection of solid organs such as kidney, liver, heart, lung or cornea, as well as rejection of bone marrow transplants including graft-versus-host disease (GVHD)), or from localized chronic or acute autoimmune or allergic reactions. Autoimmune diseases include acute glomerulonephritis; rheumatoid or reactive arthritis; chronic glomerulonephritis; inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, and some forms of diabetes, or any other autoimmune state where attack by the subject's own immune system results in pathologic tissue destruction. Allergic reactions include allergic asthma, chronic bronchitis, acute and delayed hypersensitivity. Systemic inflammatory disease states include inflammation associated with trauma, burns, reperfusion following ischemic events (e.g. thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques. In a particular embodiment, the immunological disease is sepsis.

Cancer Therapy Applications

Another aspect of the present invention is directed to methods of predicting the onset of, or detecting an increased susceptibility to, an inflammatory disease in a patent receiving a cancer treatment. In general, the methods comprising determining the presence of a neutrophilic surface protein in a biological sample of the patent, and predicting based upon such determination.

The detection and assessment described herein may be carried out prior to, during, and subsequent to an active treatment, and preferably over an entire cancer treatment regime or period. Generally speaking, a range of cancer therapies may be involved, but in most respects the treatment of a patient may be undertaken to decrease or limit the pathology caused by a cancer harbored in the individual. Cancer treatments include, but are not limited to a) administration of a composition, such as a pharmaceutical composition, and/or b) administration of radiation therapy, each of which may be performed either prophylactically, subsequent to the initiation of a pathologic event or contact with an etiologic agent, and optionally in combination with c) administration of a surgical procedure (such as lumpectomy or modified radical mastectomy).

Examples of cancers include lymphomas, carcinomas and hormone-dependent tumors (e.g., breast, prostate or ovarian cancer). Abnormal cellular proliferation conditions or cancers that may be treated in either adults or children include solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

Particular examples of chemotherapeutic agents that may be involved in a patient's cancer therapy include, for instance adriamycin, aldesleukin, allopurinol, altretamine, amifostine, anastrozole, asparaginase, betamethasone, bexarotene, bicalutamide, bleomycin, busulfan, capecitabine, carboplatin, cannustine, chlorambucil, cisplatin, cladarabine, conjugated estrogen, cortisone, cyclophosphamide, cylarabine, dacarbazine, daunorubicin, dactinomycin, denileukin, dexamethasone, discodermolide, docetaxel, doxorubicin, eloposidem, epirubicin, epoetin, epothilones, estramustine, esterified estrogen, ethinyl estradiol, etoposide, exemestane, flavopirdol, fluconazole, fludarabine, fluorouracil, flutamide, floxuridine, gemcitabine, gemtuzumab, goserelin, hexamethylmelamine, hydrocortisone, hydroxyurea, idarubicin, ifosfamide, interferon, irinotecan, lemiposide, letrozole, leuprolide, levamisole, levothyroxine, lomustine, mechlorethamine, melphalan, mercaptopurine mechlorethamine, megesterol, methotrexate, methylprednisolone, methyltestosterone, mithramycin, mitomycin, mitotane, mitoxantrone, mitozolomide, mutamycin, nilutamide, paclitaxel, pamidronate, pegaspargase, pentostatin, plicamycin, porfimer, prednisolone, procarbazine, rituximab, sargramostim, semustine, skeptozocin, tamoxifien, temozolomide, teniposide, testolactone, thioguanine, thiotepa, tomudex, topotecan, toremifene, trastumuzab, tretinoin, semustine, skeptozolocin, valrubicin, verteporfin, vinblastine, vincristine, vindesine, vinorelbine and their salts. Exemplary radiation therapies include conventional techniques employing ionizing radiation to control malignant cells (often referred to in the art as X-irradiation or XRT).

Computer Systems and Computer Program Products

To determine and identify sequence identities, structural homologies, motifs and the like in silico, the methods and techniques of the invention can be stored, recorded, and manipulated on any medium which can be read and accessed by a computer. Accordingly, the invention provides computers, computer systems, computer readable mediums, computer programs products and the like recorded or stored thereon including one or more instructions for carrying out the methods described herein. As used herein, the words “recorded” and “stored” refer to a process for storing information on a computer medium. A skilled artisan can readily adopt any known methods for recording information on a computer readable medium to generate manufactures comprising one or more of methods of the invention, or discrete steps thereof.

Another aspect of the invention is a computer readable medium having recorded thereon instructions or programs for carrying out one or more steps of the methods described herein. Computer readable media include magnetically readable media, optically readable media, electronically readable media and magnetic/optical media. For example, the computer readable media can be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

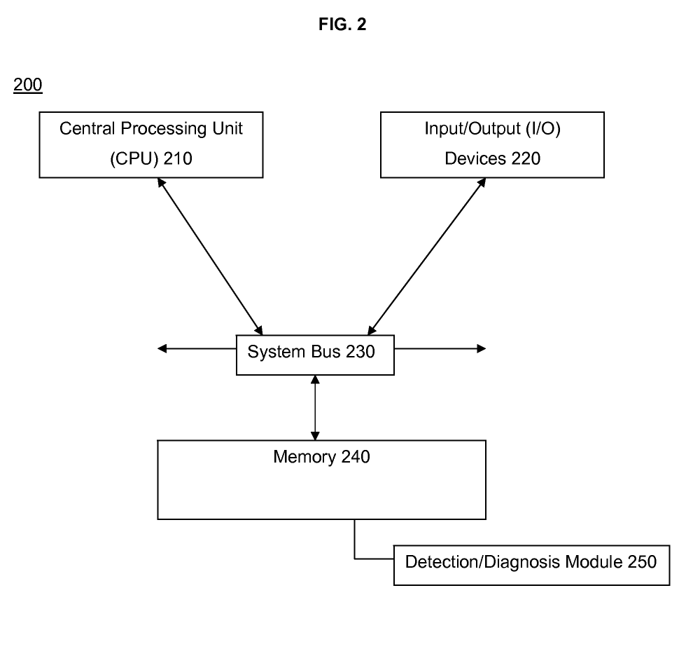

In preferred embodiment, at least one step of the methods of the present invention is performed using a computer as depicted in FIG. 2. FIG. 2 generally illustrates a computer for implementing selected operations associated with the methods of the present invention. The computer 200 includes a central processing unit 210 connected to a set of input/output devices 220 via a system bus 230. The input/output devices 220 may include a keyboard, mouse, scanner, data port, video monitor, liquid crystal display, printer, and the like. A memory 240 in the form of primary and/or secondary memory is also connected to the system bus 230. These components of FIG. 2 generally characterize a standard computer. This standard computer is programmed in accordance with the invention. In particular, the computer 200 can be programmed to perform various operations of the methods of the present invention.

The memory 240 of the computer 200 may store a detection/diagnosis module 250. Stated differently, the detection/diagnosis module 250 can perform the operations associated with the steps described herein (for example, one or more of steps 120, 130, and 140 of FIG. 1). The detection/diagnosis module may include devices and/or capabilities for performing tasks or steps described herein including, but not limited to, analyzing one or more biological substances, identifying the biological substance, and diagnosing the disease or susceptibility to the disease after the identification. The executable code of the detection/diagnosis module 250 may utilize any number of numerical techniques to perform the diagnosis.

Kits

The invention also provides kits. By way of example only and in no way limiting, the kit may include a needle and syringe or other sample collection implement and a detector medium for the detection of a biological substance in the sample. The kit may also include written instructions. In preferred embodiments, the kit may include bottles including reagents for use in detection of the biological substance. Exemplary reagents include those that may be used in one or more of the detection methods described herein, such as immunoassays. For instance, the reagents may include one or more substances that specifically hybridize to nucleic acids encoding particular proteins or markers (including probes and primers of the proteins), and reagents that specifically bind to the proteins, e.g., antibodies, that may be used to detect the presence or absence of the biological substance.

Suitable packaging and additional articles for use (e.g., measuring cups, well plates, and the like) are known in the art and may be additionally be included.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

Radiation exposure can occur on the large scale, such as the nuclear attack on Hiroshima and the nuclear accident in Chernobyl, and on the small scale, such as daily exposure to radioactive substances in the laboratory. Lethal dosages of radiation result in death while sub-lethal dosages induces neutropenia, an immune compromised condition characterized by having less than 5×10⁵ neutrophils/ml of blood. Neutropenia predisposes to sepsis; Measuring the amount of sCD16b in an irradiated patient's blood and better assess who can benefit from immediate medical attention to prevent sepsis.

A simple screening procedure is needed to quickly and accurately identify these individuals. One common and established procedure involves assessing blood polymorph nuclear neutrophil (PMN) concentration by testing for the number of neutrophils in a person's blood stream. However, this method is not adequate because it only assesses the number of neutrophils in the blood, disregarding those in bone marrow, tissues, and elsewhere in the body (Huizinga et al., 1990). Human neutrophils express an Fc isoform, CD16b or FcγRIII that is glycosylphosphatidylinositol (GPI)-anchored and spontaneously shed when the linkage is cleaved. Soluble CD16b (sCD16b) concentration in the blood may be a better indicator of the total neutrophil concentration in the whole body. CD16b is easily accessible in bodily fluids: serum, plasma, seminal matter, and saliva. We developed a sensitive ELISA to measure the amount of SCD16b in blood. Heretofore, the biological roles of linked and shed CD16 have not been adequately examined. We have taken advantage of the fact that phosphatidylinositol-specifc phospholipase C (PI-PLC) is an enzyme known to cleave the phosphoglycerol bond found in GPI-anchored proteins (Edberg et al., 1991) that induces the shedding of CD16b on freshly isolated human neutrophils. We also examined the effects of other cytokines and chemokines on CD16b shedding in the supernatants of cultured human neutrophils. To better understand the function of CD16b, we studied the effects of shedding CD16b, by various agents on neutrophil bacterial killing efficiency and migration through fibrin gels.

As a first step in establishing this ELISA for distinguishing neutropenic patients who are likely to become septic from neutropenic patients who are not in such danger, we will use an animal model developed by Dr. Tanya Mayadas of Harvard University Medical School. Dr. Mayadas has created a transgenic mouse strain whose neutrophils express the human form of CD16b (4). We have confirmed that CD16b is not shed by mouse immune cells and is not present in mouse blood plasma. We are in the process of collecting samples from healthy human volunteers to determine the range of CD16b in the blood of normal individuals and the concentration of CD16 on their neutrophils.

The next step is to compare sCD16b levels in blood plasma of sham-irradiated and sub-lethally X-irradiated transgenic mice. We will monitor the concentration of sCD16b in the blood in order to better assess the immunological potential of mice that have been exposed to sub-lethal doses of radiation. We will also correlate the blood levels of CD16b with the capacity of irradiated mice to resist bacterial infection. We will use the data from these experiments to assess whether measuring the plasma concentration of soluble CD16b is useful for distinguishing neutropenic animals in danger of fatal spontaneous or provoked sepsis from neutropenic animals that are not in such danger. Assuming we find that sCD16b in mouse blood plasma is an effective predictor of fatal sepsis, there are other surface proteins such as L-selectin and TNF-receptors found on neutrophils that are shed as well. We will examine and compare the usefulness of assessing each of these soluble proteins with than found for CD16b. It is possible that assessing several of these proteins may be a better method than CD16 alone.

If, as expected, the blood concentration of CD16b or other shed neutrophil surface proteins are indicative of the status of immune competence, we will explore the use of such a test to assess the immune status of patients undergoing X-irradiation or chemotherapy for cancer. Cancer chemotherapy, like radiation, impairs bone marrow production of neutrophils. Patients on chemotherapy often become profoundly neutropenic and must be closely monitored for signs of infection (e.g., fever). Therefore, we will examine the blood plasma concentrations of CD16b in neutropenic patients who have undergone chemotherapy. Such measurements may be better than simply monitoring the white blood cell count to assess when the immune status of a patient on chemotherapy. Such information may better enable the caring physician to determine the appropriate times to schedule chemotherapy or adjust the dose.

### Example 2

### Shedding of Fc-Gamma-IIIB Reduces Human Neutrophil Bactericidal Activity

Fcγ receptor type IIIb (FcγRIIIb, CD16b) is a GPI-anchored protein that binds the Fc domain of IgG and is highly expressed on human neutrophils. Neutrophils activated by chemoattractants, such as fMLP, shed FcγRIIIb resulting in its release into plasma and other body fluids. We have developed a sensitive ELISA assay that enables us to measure soluble FcγRIIIb in normal human plasma and found concentrations ranging from 100 ng/ml to 200 ng/ml. We have used this assay to examine the effect of chemoattractants, enzymes, and activators of neutrophil function on FcγRIIIb shedding. We report that 10⁶ human neutrophils incubated for 90 min at 37° C. with 10⁻⁷ M PMA, 10⁻⁷M fMLP, C5a (produced by incubation of IgG-opsonized S. epidermidis in human plasma), or 6.25 mUnits PI phopholipase C (an enzyme that specifically cleaves GPI-linkages), in 1 ml phosphate buffered saline containing 0.1% BSA and 5.5 mM glucose released about 5 ng of FcγRIIIb/10⁶ neutrophils. In contrast, addition of 10⁻⁷M LTB4 did not stimulate any FcγRIIIb release above background, indicating that not all chemoattractants promote FcγRIIIb release. Neutrophils pre-treated with 6.25 mUnits of PLC and incubated for 90 min at 37° C. in stirred suspensions with 10⁵ cfu/ml human serum-opsonized S. epidermidis killed only 50% of these bacteria, compared to 98% killing by untreated neutrophils under the same conditions. Thus, chemoattractant-mediated release of FcγRIIIb from neutrophils may alter the cells' bactericidal activity.

### Example 3

### The Role of CD16B in Killing of Staphylococcus epidermis by Human Neutrophils

Fcγ receptor type IIIb (FcγRIIIb) is a receptor for the Fc region of IgG and is highly expressed on human neutrophils. FcγRIIIb is shed from these cells and is found in a soluble form in plasma and other body fluids. We have developed as accurate ELISA assay to measure the concentration of soluble FcγRIIIb in both human plasma and in the supernatants of cultured human neutrophils. We have examined some of biological properties of neutrophil shedding of this receptor in response to various chemoattractants and cytokines. The addition of 10⁻⁷ M fMLP, and 10⁻⁷ M PMA promotes a dramatic shedding of FcγRIIIb by human neutrophils. In contrast no increase in FcγRIIIb shedding was observed in 10⁻⁷ M LTB4-treated human neutrophils as compared to control cells. Phospholipase C (PLC) is a specific enzyme that cleaves GPI-linked proteins including FcγRIIIb. The addition of PLC (0.25 units/ml) maximally sheds FcγRIIIb from human neutrophils and once shed, these human neutrophils were less efficient in killing human serum opsonized S. epidermidis in suspension and less able to generate hydrogen peroxide when they adhered to fibrin-coated surfaces and stimulated with 10⁻⁷ M fMLP. Thus, FcγRIIIb plays a major role in the capacity of human neutrophils to kill S. epideridis.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

### CITED DOCUMENTS

(1) Li Y, Karlin A, Loike J D, Silverstein S C. A critical concentration of neutrophils is required for effective bacterial killing in suspension. Proc Natl Acad Sci USA. 2002 Jun. 11; 99(12):8289-94.

(2) Li Y, Karlin A, Loike J D, Silverstein S C. Determination of the critical concentration of neutrophils required to block bacterial growth in tissues. J Exp Med. 2004 Sep. 6; 200(5):613-22.

(3) Huizinga, T M, de Haas, M, van Oers, M H, Kleijer, M, Vile, H, van der Wouw, P A, Moulijn, A, van Weezel, H, Roos, D, von dem Borne, A E. The plasma concentration of soluble Fc-gamma RIII is related to production of neutrophils. Br J Haematol. 1994 July; 87(3):459-63.

(4) Coxon A, Cullere X, Knight S, Sethi S, Wakelin M W, Stavrakis G, Luscinskas F W, Mayadas T N. Fc gamma RIII mediates neutrophil recruitment to immune complexes, a mechanism for neutrophil accumulation in immune-mediated inflammation. Immunity. 2001 June; 14(6):693-704.

What is claimed is:

1. An enzyme linked immunosorbent assay (ELISA) kit for determining the presence of soluble CD16b protein, which comprises at least one antibody specific to the protein to measure its molar ratio in a biological sample of a neutropenic patient, and wherein said molar ratio is indicative of the onset or susceptibility of sepsis in the patient.

2. A method of predicting the onset of sepsis in a neutropenic patient, the method comprising providing a biological sample from the patient; determining the presence of soluble CD16b protein in the biological sample; and predicting the onset of sepsis based on the presence or absence of soluble CD16b in the biological sample, wherein the determining is carried out using an ELISA kit in accordance with claim 1.

3. A method of detecting an increased susceptibility to sepsis in a neutropenic patient, the method comprising providing a biological sample from the patient; determining the presence of soluble CD16b protein in the biological sample; and predicting the onset of sepsis based on the presence or absence of soluble CD16b in the biological sample, wherein the determining is carried out using an ELISA kit in accordance with claim 1.

4. A method of predicting the onset of sepsis in a neutropenic patient receiving a cancer treatment, the method comprising providing a biological sample from the patient; determining the presence of soluble CD16b protein in the biological sample; and predicting the onset of sepsis based on the presence or absence of soluble CD16b in the biological sample.

5. A method of detecting an increased susceptibility to sepsis in a patient receiving a cancer treatment, the method comprising analyzing a biological sample from the patient for the presence of soluble CD16b protein, wherein the presence or absence of soluble CD16b protein is indicative of an increased susceptibility to sepsis.

6. A method of predicting the onset of an inflammatory disease in a neutropenic patient, the method comprising providing a biological sample from the patient; determining the presence of a neutrophilic surface protein in the biological sample; and predicting, based on said determining, the onset of the inflammatory disease in the neutropenic patient.

7. A method of detecting an increased susceptibility to an inflammatory disease in a neutropenic patient, the method comprising analyzing a biological sample from the patient for the presence of a neutrophilic surface protein, wherein the presence of the neutrophilic surface protein is indicative of an increased susceptibility to the inflammatory disease.

8. A method of predicting the onset of an inflammatory disease in a patient receiving a cancer treatment, the method comprising providing a biological sample from the patient; determining the presence of a neutrophilic surface protein in the biological sample; and predicting, based on said determining, the onset of the inflammatory disease in the patient.

9. A method of detecting an increased susceptibility to an inflammatory disease in a patient receiving a cancer treatment, the method comprising analyzing a biological sample from the patient for the presence of a neutrophilic surface protein, wherein the presence of the neutrophilic surface protein is indicative of an increased susceptibility to the inflammatory disease.

10. The method of any one of claims 6-9 wherein the inflammatory disease is sepsis.

11. The method of claim 4, 5, 8 or 9 wherein the cancer treatment is a radiation therapy or a chemotherapy.

12. The method of claim 1 or 2 wherein the neutrophilic surface protein is soluble CD16b.

13. The method of any one of claims 6-9 wherein the neutrophilic surface protein is other than soluble CD16b.

14. The method of any one of claims 6-9 wherein the neutrophilic surface protein is a shed neutrophilic surface protein.

15. The method of any one of claims 6-9 wherein the neutrophilic surface protein is L-selectin or a TNF-receptor.

16. The method of any one of claims 6-9 wherein the step of determining the presence of a neutrophilic surface protein is carried out by an immunoassay.

17. The method of claim 16 wherein the immunoassay is an enzyme-linked immunosorbent assay (ELISA).

18. The method of claim 17 wherein the determining is carried out using an ELISA kit in accordance with claim 1.

19. The ELISA kit of claim 1 wherein the at least one antibody is an anti-CD16b polyclonal or monoclonal antibody.

20. The ELISA kit of claim 1 wherein the biological sample is blood or plasma from the patient.
